(19) United States
(12) Patent Application Publication
(10) Pub. No.: US 2009/0069631 A1
(43) Pub. Date: Mar. 12, 2009

(54) FLEXIBLE TUBE FOR AN ENDOSCOPE

(75) Inventor: Hitoshi Ogura, Higashiyamato-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/913,143

(22) PCT Filed: May 12, 2006
(86) PCT No.: PCT/JP2006/309590

§ 371 Date: Oct. 30, 2007

(30) Foreign Application Priority Data
May 12, 2005 (JP) 2005-139772

(51) Int. Cl. A61B 1/00

(52) U.S. Cl. 600/121

(57) ABSTRACT

A flexible tube for an endoscope, which has a surface coated with an outer covering, wherein the outer covering is formed of a material comprising a polyolefin thermoplastic elastomer. The flexible tube is excellent not only in resistance to a washing liquid, a sterilizing liquid and a high-pressure steam sterilization but also in impact resilience, the flexible tube also having appropriate flexibility.

### TECHNICAL FIELD

This invention relates to a flexible tube for an endoscope, and, in particular, to a flexible tube for an endoscope, which is excellent in chemical resistance and in autoclave resistance.

### BACKGROUND ART

The flexible tube for an endoscope is generally covered with a flexible outer covering. This outer covering acts not only to facilitate the insertion of the flexible tube into a body cavity of the human body but also to prevent liquid such as a body fluid from permeating into the interior of the flexible tube.

As for the resin constituting the outer covering of the flexible tube for an endoscope, polyurethane elastomer has been conventionally employed in general. However, the employment of polyurethane elastomer as an outer covering of the flexible tube for an endoscope is accompanied with a problem that polyurethane elastomer is incapable of withstanding the high-pressure steam sterilization method using an autoclave, which is recently attracting much attention as a method for sterilizing the endoscope (see JP-A 2001-346754 [KOKAI]). Namely, there is a problem that, when the high-pressure steam sterilization method is applied to the aforementioned flexible tube, the tensile strength of the outer covering is caused to deteriorate.

Although it has been studied to use, as a hard segment exhibiting autoclave resistance, polyester elastomer containing polybutylene naphthalate, etc., for forming the outer covering of the flexible tube for an endoscope, the resultant flexible tube having such an outer covering is defective in that it becomes too high in flexibility to use it for an elongated endoscope for inspecting the large intestine (see JP-A 2002-311536 [KOKAI]).

The present invention has been made in view of the aforementioned circumstances and hence, the purpose of the present invention is to provide a flexible tube for an endoscope, which is excellent not only in resistance to a washing liquid, a sterilizing liquid and the high-pressure steam sterilization but also in impact resilience, the flexible tube also having appropriate flexibility.

### DISCLOSURE OF INVENTION

According to one aspect of the present invention, there is provided a flexible tube for an endoscope, which has a surface coated with an outer covering, wherein the outer covering is formed of a material comprising a polyolefin thermoplastic elastomer.

Since the flexible tube for an endoscope, which is constructed as described above according to the present invention, is covered with a polyolefin thermoplastic elastomer on its surface, it is now possible to enable the flexible tube to exhibit excellent chemical resistance and excellent autoclave resistance. Furthermore, this flexible tube for an endoscope is enabled to retain excellent insertability for a long period of time and to exhibit excellent impact resilience. Additionally, since this flexible tube for an endoscope has appropriate flexibility, the insertability of the flexible tube can be enhanced, thus providing excellent effects that the burden (pain) on a patient can be reduced.

### BRIEF DESCRIPTION OF DRAWINGS

FIG. 1 is a cross-sectional view illustrating the flexible tube for an endoscope according to one embodiment of the present invention; and

FIG. 2 is a cross-sectional view illustrating the flexible tube for an endoscope according to another embodiment of the present invention.

### BEST MODE FOR CARRYING OUT THE INVENTION

Next, the various embodiments of the present invention will be explained with reference to drawings.

The flexible tube for an endoscope according to one embodiment of the present invention is characterized in that it is covered with an outer covering formed of a polyolefin thermoplastic elastomer. In the case of the flexible tube for an endoscope which is constructed as described above, since the polyolefin thermoplastic elastomer is excellent in heat resistance and in resistance to hydrolysis and also excellent in resistance to a washing liquid, a sterilizing liquid and the high-pressure steam sterilization, the flexible tube for an endoscope, having the outer covering is formed of such an elastomer is enabled to exhibit excellent chemical resistance as well as excellent autoclave resistance.

In the case of the flexible tube for an endoscope according to the present invention, a coating layer may be formed on the surface of the outer covering by coating a fluorine resin coating material thereon in order to enhance the insertability thereof and to prevent the degradation thereof. However, since the polyolefin thermoplastic elastomer constituting the outer covering is a material which is inherently poor in adhesiveness and hence low in adhesion to the fluorine resin coating material, it may be advisable to apply a coating of primer between the outer covering and the coating layer.

As for the primer, it is possible to preferably employ chlorinated polyolefin, especially, maleic modified or acrylic modified chlorinated polyolefin.

Incidentally, instead of applying the coating of primer to an interface between the outer covering and the fluorine resin coating material, the primer may be incorporated in advance into the polyolefin thermoplastic elastomer to be used for constituting the outer covering, thus obtaining almost the same effects as described above.

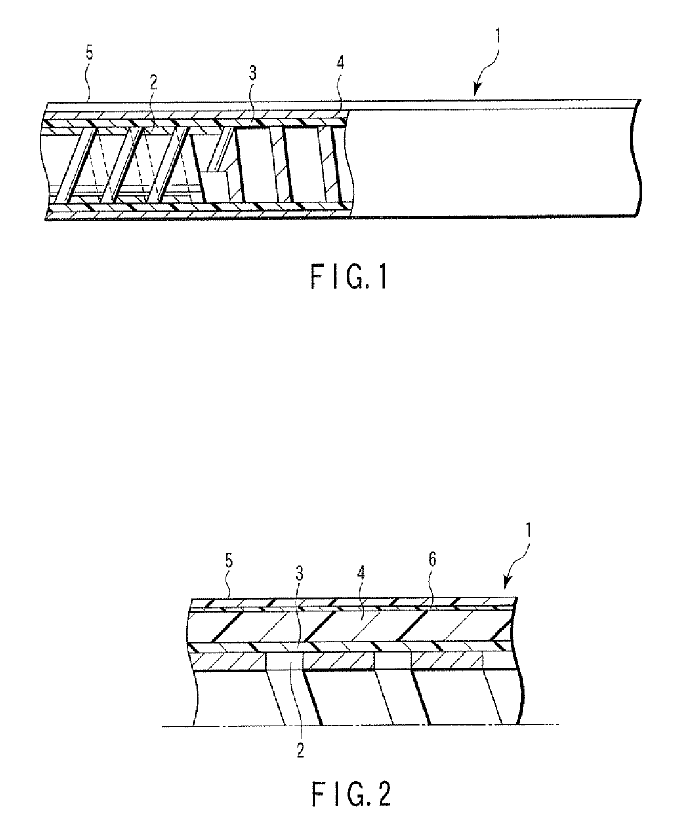

FIG. 1 is a cross-sectional view illustrating the flexible tube 1 for an endoscope according to one embodiment of the present invention.

As shown in FIG. 1, the flexible tube 1 for an endoscope (hereinafter referred to simply as “the flexible tube 1”) is constituted by a spiral tube 2, a mesh tube 3 entirely covering the outer surface of the spiral tube 2, and an outer covering 4 entirely covering the outer surface of the mesh tube 3. Incidentally, the outer circumferential wall of the outer covering 4 is covered with a coating layer 5.

As for the material for constituting the spiral tube 2, it is possible to employ stainless steel or a copper alloy. The mesh tube 3 may be fabricated by knitting a plurality of metallic or non-metallic fine wires into a tubular structure. As for the material for the fine wire, it is possible to employ a metallic material such as stainless steel or a non-metallic material such as plastic. Further, in order to enhance the adhesion of the mesh tube 3 to the resin constituting the outer covering, the metallic fine wire may be employed in combination with the non-metallic fine wire.

The outer covering 4 covering the outer surface of the mesh tube 3 is constituted by a polyolefin thermoplastic elastomer. This polyolefin thermoplastic elastomer is formed of a mixture comprising, as a hard segment, polyolefin such as polyethylene or polypropylene, and, as a soft segment, olefin rubber. Since the polyolefin thermoplastic elastomer is formed of a mixture as described above, it is possible to obtain an elastomer exhibiting desired flexibility by suitably adjusting the ratio of the olefin rubber.

The polyolefin thermoplastic elastomer can be generally classified into three types according to the blending methods, i.e., a simple blend type; a dynamic cross-linking type wherein a rubber moiety is blended therein while allowing the rubber moiety to cross-link, thus enabling cross-linked rubber to be finely blended therein; and a polymerization type wherein a comonomer constituting the soft segment is incorporated therein concurrent with the polymerization of propylene constituting the hard segment, thus blending the comonomer therein while carrying out the polymerization thereof.

As a result of extensive studies made by the present inventors on these types of polyolefin thermoplastic elastomer, it has been found out that, among the aforementioned dynamic cross-linking type, a complete dynamic cross-linking type wherein the rubber moiety is completely cross-linked and the aforementioned polymerization type are relatively low in deterioration of mechanical strength even if they are subjected to the sterilization treatment by means of various kinds of chemicals or by means of an autoclave, thus exhibiting excellent resistance to these sterilization treatments.

Since the polyolefin thermoplastic elastomer of this complete dynamic cross-linking type has no reactive group in its molecular structure, it is assumed that it can hardly deteriorate as a result of these sterilization treatments. As for the polyolefin thermoplastic elastomer of the polymerization type, it is assumed that, since the soft segment is dispersed on the occasion of polymerization in this case, the soft segment is finely dispersed in the hard segment as compared with the elastomer of other types, thereby enabling the elastomer to hardly deteriorate.

As for the film thickness of the outer covering 4, although there is not any particular limitation, it is generally preferable to confine it to the range of 0.5-1.5 mm or so.

The coating layer 5 for covering the outer circumference of the outer covering 4 may be formed by coating a fluorine resin coating material exhibiting excellent gas barrier properties on the outer circumference of the outer covering 4. The provision of this coating layer 5 is effective in enhancing the insertability of the flexible tube and in suppressing the deterioration of the outer covering 4 that may be caused to occur due to chemicals. The coating material to be employed for forming this coating layer 5 is a two-part reactive paint containing a base resin and a curing agent. As for the essential component of the base resin, it is possible to employ a fluorine resin-containing copolymer which is excellent in solubility to a solvent. Further, this fluorine-containing copolymer may contain hydroxyl group in its molecule.

As for the essential component of the curing agent, it is possible to employ isocyanate. As for the specific example of this isocyanate, it includes hexamethylene diisocyanate having an active isocyanate group on its terminal.

The two-part reactive paint containing these components can be coated on the outer surface of outer covering 4 by means of spraying, brushing, roller coating, dipping, etc. In this embodiment, the coating may be appropriately carried out by means of dipping. Upon finishing the coating of the two-part reactive paint, the coated layer is left standing in a heated atmosphere of 60-100° C., for example 80° C., for a period of 300-900 minutes, for example 600 minutes, to thereby allow the two-part reactive paint to cure, thus forming the coating layer 5.

Although there is not any particular limitation with respect to the thickness of the coating layer 5, it is generally preferable to confine the film thickness of the coating layer 5 to the range of 5-100 μm or so.

Incidentally, since the polyolefin thermoplastic elastomer is inherently poor in adhesiveness, it is not likely that a fluorine resin coating material can easily adhere to the surface of outer covering 4. In that case, it is advisable to interpose a primer layer 6 between the outer covering 4 and the coating layer 5, thereby making it possible to for the coating layer 5 which is excellent in adhesion.

As for the primer layer 6, chlorinated polyolefin can be effectively employed. Especially, by making use of maleic modified or acrylic modified primer, it is possible to realize excellent adhesion of the coating layer.

Further, there are two types of primer, i.e., a solvent-type primer and an aqueous-type primer. Among them, since the solvent-type primer contains no adhesion-obstructing component such as an emulsifier, etc., in contrast to the aqueous-type primer, the solvent-type primer is enabled to exhibit excellent adhesion and hence more preferable for use.

The primer layer can be formed by coating a primer by means of spraying, brushing, roller coating, dipping, etc. In this embodiment, the coating may be appropriately carried out by means of dipping. Upon finishing the formation of the primer layer 6, the primer layer 6 is left standing at a temperature of 20-100° C., for example 25° C. for a period of 10-60 minutes, for example 30 minutes to thereby allow the primer layer 6 to cure. Subsequently, the aforementioned fluorine resin coating material is coated on the surface of primer layer 6 to form the coating layer 5.

The aforementioned primer component may not necessarily be coated on the surface of outer covering 4 as described above. Namely, the primer component may be directly kneaded together with the polyolefin thermoplastic elastomer to be employed as a material for the outer covering 4. By doing so, it is possible to enhance the adhesion of the coating layer likewise as described above.

In the case of the flexible tube 1 for an endoscope which is constructed according to one embodiment of the present invention, since the outer covering 4 is constituted by a polyolefin thermoplastic elastomer, it is possible to realize excellent resistance of the flexible tube 1 to sterilizing liquids as well as to the autoclave sterilization.

Next, there will be explained specific examples and comparative examples each explaining the manufacture of a flexible tube for an endoscope, wherein the mesh tube is covered with various kinds of resin as an outer covering.

### EXAMPLE 1

By making use of an extruder, the surface of the mesh tube was covered with a complete cross-linking-type polyolefin thermoplastic elastomer (Sarlink 4000 Series: DSM Co., Ltd.; Santoplane: AES Co., Ltd.) to form an outer covering, thus manufacturing a flexible tube for an endoscope.

### EXAMPLE 2

By making use of an extruder, the surface of the mesh tube was covered with a polymerization-type polyolefin thermoplastic elastomer (Exelene: Sumitomo Chemicals Industries) to form an outer covering, thus manufacturing a flexible tube for an endoscope.

### EXAMPLE 3

The surface of the outer covering of the flexible tube that had been manufactured in Example 1 was subjected to a primer application treatment by making use of chlorinated polyolefin (Hardrene: Toyo Kasei Co., Ltd.) and then subjected to a coating treatment by making use of a fluorine resin coating material.

### EXAMPLE 4

The surface of the outer covering of the flexible tube that had been manufactured in Example 2 was subjected to a primer application treatment by making use of acrylic modified chlorinated polyolefin (Hardrene: Toyo Kasei Co., Ltd.) and then subjected to a coating treatment by making use of a fluorine resin coating material.

### EXAMPLE 5

The surface of the outer covering of the flexible tube that had been manufactured in Example 2 was subjected to a primer application treatment by making use of maleic modified chlorinated polyolefin (Hardrene: Toyo Kasei Co., Ltd.) and then subjected to a coating treatment by making use of a fluorine resin coating material.

### EXAMPLE 6

By making use of an extruder, the surface of the mesh tube was covered with a dry-blended mixture containing 100 parts by weight of a complete cross-linking-type polyolefin thermoplastic elastomer and 3 parts by weight of chlorinated polyolefin and then subjected to a coating treatment by making use of a fluorine resin coating material, thus manufacturing a flexible tube for an endoscope.

### COMPARATIVE EXAMPLE 1

By making use of an extruder, the surface of the mesh tube was covered with a polyurethane elastomer (E372: Nippon Milactran Co., Ltd.) to form an outer covering and then subjected to a coating treatment by making use of a fluorine resin coating material, thus manufacturing a flexible tube for an endoscope.

### COMPARATIVE EXAMPLE 2

By making use of an extruder, the surface of the mesh tube was covered with a polyester elastomer (Hytrel: Tohre-Du Pont Co., Ltd.) to form an outer covering and then subjected to a coating treatment by making use of a fluorine resin coating material, thus manufacturing a flexible tube for an endoscope.

The chemical resistance, autoclave resistance, flexibility and insertability of the flexible tubes obtained in the aforementioned Examples and Comparative Examples were evaluated.

The evaluation of chemical resistance was performed by a process wherein the flexible tube was immersed in a 30% solution of peracetic acid for 3000 minutes and the tensile strength of the flexible tube was measured before and after the process, this process being repeated to 600 samples to measure the ratio of deterioration in tensile strength, thereby evaluating the chemical resistance. Further, the autoclave resistance was evaluated by a process wherein the flexible tube was placed in a high-pressure steam atmosphere (135° C., 2 atm.) in an autoclave for 3000 minutes and the tensile strength of the flexible tube was measured before and after the process, this process being repeated to 600 samples to measure the ratio of deterioration in tensile strength, thereby evaluating the autoclave resistance. The criterion of these evaluations was as follows.

When the ratio of deterioration was not more than 5%: ⊚

When the ratio of deterioration was not more than 10%: ◯

When the ratio of deterioration was not more than 20%: X

When the ratio of deterioration was more than 20%: X

The flexibility of the flexible tube was measured according to the hardness thereof that could be determined through the feeling of hand. The insertability was evaluated by measuring the frictional coefficient. The criterion of these evaluations was as follows.

⊚: Excellent

◯: Good

Δ: Fair

X: Bad

The results of evaluation on these Examples and Comparative Examples are summarized in the following Table 1.

TABLE 1

| | Chemical | | | | |
|---|---|---|---|---|---|
| | (peracetic | | | | |
| | aid) | Autoclave | | Insertion | |
| | resistance | resistance | Flexibility | property | Remarks |
| Ex. 1 | ⊚ | ◯ | ⊚ | ◯ | |
| Ex. 2 | ⊚ | ◯ | ⊚ | ◯ | |
| Ex. 3 | ⊚ | ⊚ | ⊚ | ⊚ | |
| Ex. 4 | ⊚ | ⊚ | ⊚ | ⊚ | |
| Ex. 5 | ⊚ | ⊚ | ⊚ | ⊚ | |
| Ex. 6 | ⊚ | ⊚ | ⊚ | ⊚ | |
| Comp. | Δ | X | ◯ | ⊚ | Not resistive |
| Ex. 1 | | | | | to chemicals/ |
| | | | | | autoclave |
| Comp. | Δ | X | X | ⊚ | Not resistive |
| Ex. 2 | | | | | to chemicals/ |
| | | | | | autoclave |

It will be recognized from above Table 1 that all of the flexible tubes for an endoscope according to Examples 1-6 wherein a polyolefin thermoplastic elastomer was employed for forming the outer covering exhibited excellent effects in all respects including the chemical resistance, autoclave resistance, flexibility and insertability.

Whereas, in the case of Comparative Example 1 where polyurethane elastomer was employed for forming the outer covering, the flexible tube was inferior in autoclave resistance, and in the case of Comparative Example 2 where polyester elastomer was employed for forming the outer covering, the flexible tube was inferior in autoclave resistance and flexibility as shown in Table 1.

It should be understood that the present invention is not limited to the aforementioned embodiments but can be variously modified in carrying out the present invention without departing from the gist of the present invention.

1. A flexible tube for an endoscope, which has a surface coated with an outer covering, wherein the outer covering is formed of a material comprising a polyolefin thermoplastic elastomer.

2. The flexible tube for an endoscope according to claim 1, wherein the polyolefin thermoplastic elastomer is formed of a mixture comprising, as a hard segment, polyolefin, and, as a soft segment, olefin rubber.

3. The flexible tube for an endoscope according to claim 1, wherein the polyolefin thermoplastic elastomer is of a complete dynamic cross-linking type or of a polymerization type.

4. The flexible tube for an endoscope according to claim 1, wherein the outer covering has a film thickness ranging from 0.1 to 1.5 mm.

5. The flexible tube for an endoscope according to claim 1, which further comprises a coating layer on the outer covering, the coating layer being formed by applying fluorine resin coating material to the outer covering.

6. The flexible tube for an endoscope according to claim 5, wherein the fluorine resin coating material is formed of a two-part reactive paint comprising a base resin formed of a fluorine-containing copolymer, and a curing agent formed of isocyanate.

7. The flexible tube for an endoscope according to claim 5, wherein the coating layer has a film thickness ranging from 5 to 100 μm.

8. The flexible tube for an endoscope according to claim 5, which further comprises a primer layer formed between the outer covering and the outer covering.

9. The flexible tube for an endoscope according to claim 8, wherein the primer layer is formed of chlorinated polyolefin.

10. The flexible tube for an endoscope according to claim 9, wherein the primer layer is formed of maleic modified chlorinated polyolefin.

11. The flexible tube for an endoscope according to claim 9, wherein the primer layer is formed of acrylic modified chlorinated polyolefin.

12. The flexible tube for an endoscope according to claim 5, wherein the outer covering contains a primer component.
